(19) United States
(12) Patent Application Publication
(10) Pub. No.: US 2024/0423900 A1
(43) Pub. Date: Dec. 26, 2024

(54) ANHYDROUS BARRIER AND ANTIOXIDANT COMPOSITIONS

(71) Applicant: Baek Clinical Inc., Los Angeles, CA (US)

(72) Inventor: Jihoon P. Baek, Los Angeles, CA (US)

(21) Appl. No.: 18/653,884

(22) Filed: May 2, 2024

Related U.S. Application Data
(60) Provisional application No. 63/499,699, filed on May 2, 2023.

(51) Int. Cl. A61K 8/9794; A61K 8/34; A61K 8/37; A61K 8/67; A61Q 17/00

(52) U.S. Cl. CPC A61K 8/9794; A61K 8/347; A61K 8/37; A61K 8/678; A61Q 17/00

(57) ABSTRACT

Topical formulations of an anhydrous liquid, composed of primary and secondary antioxidants, barrier-repairing agent(s), and hydrocarbon compound(s). The formulations are storage stable for an extended period of time without significant degradation as measured by a change in texture, appearance or odor, and have desirable physical properties. Topical compositions of this disclosure find use in treating or preventing a variety of cosmetic and/or dermatological conditions.

### 1. CROSS-REFERENCE TO RELATED APPLICATIONS

This application claims the benefit of U.S. Provisional Application No. 63/499,699, filed May 2, 2023, which is hereby incorporated in its entirety by reference herein.

### 2. BACKGROUND

Skin is constantly exposed to environmental aggressors that compromise its natural defenses and can result in free radical damage, dryness, irritation, and sensitivity. Such environmental aggressors include, but are not limited to, sunlight, pollution, heat, cold, and low humidity. Skin that is already compromised by such factors can often react negatively to excess water exposure, and may react to water-and water-containing topical treatments with sensations or burning or stinging.

Antioxidants play a crucial role in preserving skin's defenses against free radical damage, which is associated with a number of cosmetic and/or dermatological concerns associated with aging, including skin roughness, dryness, hyperpigmentation, uneven skin tone, and wrinkling (insert citation).

Lipids and other barrier-supporting agents play a crucial role in preserving skin's barrier function, which prevents moisture loss from skin, or transepidermal water loss (TEWL), and abnormalities or deterioration of them are implicated in a range of cosmetic and/or dermatological conditions associated with dry and scaly skin, such as dermatitis, psoriasis, xerosis, ichthyosis, and eczema (insert citation). In conditions in which skin barrier function has been severely compromised, exposure to water or water-containing products can result in sensations of burning and/or stinging. Indeed, in certain dry skin conditions, reducing exposure to water is a method in which further damage to barrier function is prevented while giving time to skin to replenish its endogenous pool of lipids to restore normal barrier function (insert citation).

Anhydrous lipidic compositions have often come in the form of ointments and balms, which are often perceived as being “heavy” or “sticky” when applied to skin, especially that of the face. Said compositions often rely on materials such as petrolatum, fatty alcohols, and various waxes, which are semi-solid to solid at room temperature and contribute to the “heavy” sensorial experience of the final composition. Additionally, current anhydrous lipidic compositions include triglycerides of plant oils in their compositions, which result in the composition having a short shelf-life due to degradation of the plant oils, and also result in a foul odor over time. Facial oil compositions that include plant oils are typically not sustainable for longer shelf-life products.

An alternative vehicle would be anhydrous, oily, low-viscosity (at room temperature) compositions, often referred to as “facial oils”. These compositions are often more sensorially pleasing than ointments or balms with plant-based oils, as they are perceived to be “lighter” in texture, but vary greatly in performance. There has been and remains a need for anhydrous, low-viscosity non-sticky, topical formulations that provide relief for dry skin and measurably increase skin's defenses against free radical damage.

### 3. SUMMARY OF THE INVENTION

The present disclosure includes compositions and methods for treating, preventing, or improving dermatocosmetic conditions.

Topical formulations of an anhydrous liquid, composed of a primary oil-soluble antioxidant, secondary oil-soluble antioxidant, barrier-repairing agent, and hydrocarbon-based emollient are provided. The formulations contain antioxidants, barrier-repairing agents, and hydrocarbon-based emollients and have desirable physical properties, such as, but not limited to: increased barrier function and repair on the skin. The topical formulation is also formulated for increasing shelf-life of the product. The topical formulations can include concentrations of a primary oil-soluble antioxidant of 0.5% to 3% by weight, a secondary oil-soluble antioxidant of 0.5% to 5% by weight, a barrier-repairing agent of 4% to 30% by weight, and a hydrocarbon-based emollient of 4% to 95% by weight. The topical formulations contain less than 1% of water by weight. Topical compositions of this disclosure find use in treating or preventing a variety of cosmetic and/or dermatological conditions.

### 4. DETAILED DESCRIPTION OF THE INVENTION

This disclosure provides topical formulations of anhydrous fluids comprising a primary antioxidant, secondary antioxidant, barrier-repairing agents, and hydrocarbon-based emollients. The formulations provide moisturization for dry skin conditions and an increase in skin's antioxidant defenses. This disclosure provides particular topical formulations which have been developed and optimized to provide skin compatibility, desirable physical properties, and longer shelf-life then commercially available topical formulations that contain plant-based oils.

Topical compositions of this disclosure find use in treating or preventing a variety of cosmetic and/or dermatological conditions. Non-limiting examples of dermatocosmetic conditions that may be improved by topical application of the compositions of the present disclosure include: inflammatory dermatoses (including eczema, acne, psoriasis), and xeroses (also known in the art as dry skin or pruritus).

In some embodiments, formulations of the present disclosure include the ingredients: (i) 0.5% to 3% by weight of a primary antioxidant; (ii) 0.5% to 5% by weight of a secondary antioxidant; (iii) 4% to 30% by weight of a barrier-repairing agent; and (iii) 4% to 95% by weight of a hydrocarbon-based emollient; and less than 1% by weight water.

Traditional ointments and balms are semi-solid at room temperature and suffer from undesirable physical properties, including perceived heaviness and stickiness upon application, especially to the face. Traditional oil blends containing a mixture of non-volatile and volatile plant oils are liquid at room temperature and often lack ingredients with significant barrier-repairing properties and antioxidants that can measurably increase skin's defenses against free radical damage.

Conventional facial oils often provide emollience and a pleasant sensorial experience but lack substantial barrier repair and evidence for substantial increase in antioxidant defense. The inventor of the present disclosure surprisingly discovered that by incorporating a primary antioxidant, secondary antioxidant, barrier-repairing agent and hydrocarbon-based emollient, one can combine the benefit of relief from dry skin, a significant and measurable increase in skin's antioxidant defenses, and the desirable physical properties (perceived lightness on skin, lack of stickiness) of an oil blend that does not contain plant-based oils. Through this composition, one is able to achieve the barrier-repairing properties of an ointment and the free radical defense of a dedicated antioxidant serum with the cosmetic elegance and sensorial experience of a facial oil.

The present inventors found an alternative vehicle of anhydrous, oil-based, liquid (at room temperature) compositions. These compositions are often more sensorially pleasing than ointments or balms, but vary greatly in performance. Indeed, most plant oils and esters have little evidence for either acting as occlusives to prevent TEWL or substantively aiding in the recovery of skin's barrier function. Furthermore, research points to free fatty acid exposure to skin, when not combined in a relatively narrow range of molar ratios with cholesterol and ceramides, potentially contributing to delayed repair of skin's barrier function. The present inventor surprisingly found that plant oils and other triglycerides release free fatty acids unpredictably, as this process is dependent on the microflora that is present on one's skin, which varies greatly from individual to individual. Additionally, storage stability is often an issue for such plant oils often have short shelf lives as they break down, emitting an unpleasant odor.

### 4.1. Antioxidants

embodiments, the primary oil-soluble antioxidant comprises 0.5% to 3% by weight of the primary oil-soluble antioxidant.

In certain embodiments, the primary oil-soluble antioxidant comprises bakuchiol. Bakuchiol (10309-37-2) is a natural terpenoid antioxidant.

In certain embodiments, the primary oil-soluble antioxidant comprises Curcuma longa extract.

In certain embodiments, the primary oil-soluble antioxidant comprises Bakuchiol and Curcuma longa extract. In some embodiments, the primary oil-soluble antioxidant is present in an amount in the range of 0.5 to 3%, for example, 0.5 to 2% by weight of the composition, such as 0.5 to 1% by weight, 0.5 to 2.5% by weight, e.g., about 1%, about 3%, about 1% or about 2% by weight of the composition. In some embodiments the primary oil-soluble antioxidant comprises 0.5% or more, 1% or more, 1.5% or more, 2% or more, 2.5% or more, or 3% by weight of the primary oil-soluble antioxidantcomposition.

In certain embodiments, the primary oil-soluble antioxidant comprises bakuchiol. Bakuchiol (10309-37-2) is a natural terpenoid antioxidant. In certain embodiments, the primary oil-soluble antioxidant comprises 0.1% by weight or more bakuchiol, 0.2% by weight or more bakuchiol, 0.3% by weight or more bakuchiol, 0.4% by weight or more bakuchiol, 0.5% by weight or more bakuchiol, 0.6% by weight or more bakuchiol, 0.7% by weight or more bakuchiol, 0.9% by weight or more bakuchiol, or 1% by weight or more bakuchiol.

“Bakuchiol” as used herein refers to the compound having the following formula:

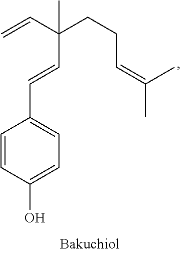 [SMILES: C=CC(C)(/C=C/c1ccc(O)cc1)CCC=C(C)C]

wherein the benzylic double-bond may be either cis or trans. As used herein, bakuchiol includes pharmaceutically acceptable salts and tautomers of bakuchiol. Phenolic compounds structurally related to bakuchiol are also included within this definition.

In certain embodiments, the primary oil-soluble antioxidant comprises Curcuma longa root extract. In certain embodiments, the primary oil-soluble antioxidant comprises 0.1% by weight or more Curcuma longa root extract, 0.2% by weight or more Curcuma longa extract, 0.3% by weight or more Curcuma longa root extract, 0.4% by weight or more Curcuma longa extract, or 0.5% by weight or more Curcuma longa root extract.

In certain embodiments, the primary oil-soluble antioxidant comprises Bakuchiol and Curcuma longa extract. In some embodiments, the primary oil-soluble antioxidant is suitable for boosting photoprotection from UVA radiation. Additional non-limiting examples of antioxidants include polydatin, phloretin, resveratrol, ferulic acid, and a mixture thereof. In some embodiments, the primary oil-soluble antioxidant can be combined with one or more UV filters, for example organic UV filters, in a cosmetically acceptable carrier. The UV filter(s) may be UVB filters, UVA filters (UVA1 and/or UVA2 filters), and/or inorganic UV filters (UVA and/or UVB filters).

In some embodiments, the primary oil-soluble antioxidant comprises a turmeric extract. In certain embodiments, the turmeric extract is selected from: turmeric, curcumin, Curcuma longa, demethoxycurcumin, bisdemethoxycurcumin, diarylheptanoids, diarylpentanoids, phenylpropenes, phenylpropenes, monoterpenes, sesquiterpenes, diterpenes, triterpenes, sterols and alkaloids. In certain embodiments, the primary oil-soluble antioxidant comprises a Curcuma longa extract.

In certain embodiments, the primary oil-soluble antioxidant comprises Bakuchiol and Curcuma longa extract.

In some embodiments, the composition comprises a secondary oil-soluble antioxidant. In certain embodiments, the secondary oil-soluble antioxidant is in an amount ranging from 0.5 to 5% by weight of the composition. In some embodiments, the secondary oil-soluble antioxidant is present in an amount in the range of 0.5 to 5%, for example, 0.5 to 4% by weight of the composition, such as 0.5 to 4.5% by weight, 0.5 to 3.5% by weight, e.g., about 1%, about 3.5%, about 1% or about 2% by weight of the composition. In some embodiments the primarysecondary oil-soluble antioxidant comprises 0.5% or more, 1% or more, 1.5% or more, 2% or more, 2.5% or more, 3% or more, 3.5% or more, 4% or more, 4.5% or more, or 5% by weight of the secondary oil-soluble antioxidantcomposition.

In certain embodiments, the secondary oil-soluble antioxidant is selected from: tocopherol, bis-ethylhexyl hydroxydimethoxy benzylmalonate, dimethylmethoxy chromanol, hexylresorcinol, and a combination thereof.

In certain embodiments, the secondary oil-soluble antioxidant is tocopherol or tocotrienol agent. In certain embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol. In certain embodiments, the secondary oil-soluble antioxidant comprises 0.1% by weight or more tocopherolbakuchiol, 0.2% by weight or more bakuchioltocopherol, 0.3% by weight or more tocopherolbakuchiol, 0.4% by weight or more bakutocopherolchiol, 0.5% by weight or more tocopherolbakuchiol, 0.6% by weight or more tocopherolbakuchiol, 0.7% by weight or more tocopherolbakuchiol, 0.9% by weight or more tocopherolbakuchiol, or 1% by weight or more tocopherolbakuchiol.

In some embodiments, the tocopherol or tocotrienol agent is present in the composition in an amount of 5% or less by weight, such as 4.5% or less, 4% or less, 3.5% or less, 3% or less, 2.5% or less, 2% or less, 1.5% or less, or 1% or less by weight of the composition.

In some embodiments of any one of the formulations described herein, the formulation excludes tocopherol or tocotrienol agents, e.g., or precursors thereof having vitamin E activity. In certain embodiments of any one of the formulations described herein, the formulation excludes vitamin E acetate.

In some embodiments, the secondary oil-soluble antioxidant is bis-ethylhexyl hydroxydimethoxy benzylmalonate (HDBM). In certain embodiments, the secondary oil-soluble antioxidant comprises 0.1% by weight or more bis-ethylhexyl hydroxydimethoxy benzylmalonate, 0.2% by weight or more bis-ethylhexyl hydroxydimethoxy benzylmalonate, 0.3% by weight or more bis-ethylhexyl hydroxydimethoxy benzylmalonate, 0.4% by weight or more bis-ethylhexyl hydroxydimethoxy benzylmalonatetocopherol, 0.5% by weight or more bis-ethylhexyl hydroxydimethoxy benzylmalonate, 0.6% by weight or more bis-ethylhexyl hydroxydimethoxy benzylmalonate, 0.7% by weight or more bis-ethylhexyl hydroxydimethoxy benzylmalonatetocopherol, 0.9% by weight or more bis-ethylhexyl hydroxydimethoxy benzylmalonate, 1% by weight or more bis-ethylhexyl hydroxydimethoxy benzylmalonate, 1.5% by weight or more bis-ethylhexyl hydroxydimethoxy benzylmalonate, or 2% by weight or more bis-ethylhexyl hydroxydimethoxy benzylmalonate.

In some embodiments, the secondary oil-soluble antioxidant is dimethylmethoxy chromanol.

In some embodiments, the secondary oil-soluble antioxidant is hexylresorcinol.

In some embodiments, the secondary oil-soluble antioxidant is a combination of tocopherol, bis-ethylhexyl hydroxydimethoxy benzylmalonate, dimethylmethoxy chromanol, and hexylresorcinol.

### 4.2. Barrier-Repairing Agent

The topical anhydrous lipid composition of the present disclosure comprises a barrier repairing agent. In some embodiments, the barrier repairing agent comprises lanolin oil, cholesterol, a cholesterol derivative, lanosterol, a lanosterol derivative, a polyglyceryl fatty acid ester, or any combination thereof.

In some embodiments, the barrier repairing agent comprises lanolin oil.

In some embodiments, the barrier repairing agent comprises cholesterol or a cholesterol derivative or analog thereof. A variety of cholesterol derivatives and analogs can be used in the compositions of this disclosure. In one embodiment, the barrier repairing agent comprises cholesterol. In one embodiment, the barrier repairing agent comprises a cholesterol derivative. In one embodiment, the cholesterol derivative is a cholesteryl ester. In one embodiment, the cholesterol derivative is a cholesteryl chloride. “Cholesteryl esters,” are cholesterol molecules having a fatty acid moiety attached through an ester bond. The ester bond is formed between the carboxylase group of the fatty acid and the hydroxyl group of cholesterol. In some embodiments, the cholesteryl ester comprises a fatty acid having from 10 to 30 carbon atoms (i.e., C₁₀-C₃₀ cholesteryl ester), such as 10 to 15, 15 to 20, 20 to 24 or 25 to 30 carbon atoms. In some embodiments, the cholesteryl ester comprises a fatty acid having 10 carbon atoms. In some embodiments, the cholesteryl ester comprises a fatty acid having 11 carbon atoms. In some embodiments, the cholesteryl ester comprises a fatty acid having 12 carbon atoms. In some embodiments, the cholesteryl ester comprises a fatty acid having 13 carbon atoms. In some embodiments, the cholesteryl ester comprises a fatty acid having 14 carbon atoms. In some embodiments, the cholesteryl ester comprises a fatty acid having 15 carbon atoms. In some embodiments, the cholesteryl ester comprises a fatty acid having 16 carbon atoms. In some embodiments, the cholesteryl ester comprises a fatty acid having 17 carbon atoms. In some embodiments, the cholesteryl ester comprises a fatty acid having 18 carbon atoms. In some embodiments, the cholesteryl ester comprises a fatty acid having 19 carbon atoms. In some embodiments, the cholesteryl ester comprises a fatty acid having 20 carbon atoms. In some embodiments, the cholesteryl ester comprises a fatty acid having 21 carbon atoms. In some embodiments, the cholesteryl ester comprises a fatty acid having 22 carbon atoms. In some embodiments, the cholesteryl ester comprises a fatty acid having 23 carbon atoms. In some embodiments, the cholesteryl ester comprises a fatty acid having 24 carbon atoms. In some embodiments, the cholesteryl ester comprises a fatty acid having 25 carbon atoms. In some embodiments, the cholesteryl ester comprises a fatty acid having 26 carbon atoms. In some embodiments, the cholesteryl ester comprises a fatty acid having 27 carbon atoms. In some embodiments, the cholesteryl ester comprises a fatty acid having 28 carbon atoms. In some embodiments, the cholesteryl ester comprises a fatty acid having 29 carbon atoms. In some embodiments, the cholesteryl ester comprises a fatty acid having 30 carbon atoms.

Example cholesteryl esters include, but are not limited to, cholesteryl oleate, cholesteryl laurate, cholesteryl myristate, cholesteryl palmitate, cholesteryl stearate, cholesteryl isostearate, cholesteryl arachidate, cholesteryl behenate, cholesteryl lignocerate, cholesteryl cerotate, cholesteryl montanate, cholesteryl melissate, and cholesteryl nonanoate.

In some embodiments, the cholesterol derivative is cholesteryl chloride, where the chloride replaces the hydroxyl group of cholesterol. In some embodiments, the cholesterol derivative is a carbonate ester of cholesterol. In some embodiments, the carbonate ester of cholesterol is cholesteryl oleyl carbonate. In some embodiments, the cholesterol derivative is cholesteryl succinate.

In some embodiments, the barrier repairing agent comprises lanosterol or a lanosterol derivative or analog thereof. A variety of lanosterol derivatives and analogs can be used in the compositions of this disclosure. In one embodiment, the barrier repairing agent comprises lanosterol. In one embodiment, the barrier repairing agent comprises a lanosterol derivative. In one embodiment, the lanosterol derivative is a lanosteryl ester. “Lanosteryl ester,” refers to a fatty acid ester of lanosterol, wherein the ester bond is formed between the carboxylate group of a fatty acid and the free hydroxyl group of lanosterol. In some embodiments, the lanosteryl ester comprises a fatty acid having from 10 to 30 carbon atoms (i.e., C₁₀-C₃₀ lanosterol ester), such as 10 to 15, 15 to 20, 20 to 24 or 25 to 30 carbon atoms. In some embodiments, the lanosteryl ester comprises a fatty acid having 10 carbon atoms. In some embodiments, the lanosteryl ester comprises a fatty acid having 11 carbon atoms. In some embodiments, the lanosteryl ester comprises a fatty acid having 12 carbon atoms. In some embodiments, the lanosteryl ester comprises a fatty acid having 13 carbon atoms. In some embodiments, the lanosteryl ester comprises a fatty acid having 14 carbon atoms. In some embodiments, the lanosteryl ester comprises a fatty acid having 15 carbon atoms. In some embodiments, the lanosteryl ester comprises a fatty acid having 16 carbon atoms. In some embodiments, the lanosteryl ester comprises a fatty acid having 17 carbon atoms. In some embodiments, the lanosteryl ester comprises a fatty acid having 18 carbon atoms. In some embodiments, the lanosteryl ester comprises a fatty acid having 19 carbon atoms. In some embodiments, the lanosteryl ester comprises a fatty acid having 20 carbon atoms. In some embodiments, the lanosteryl ester comprises a fatty acid having 21 carbon atoms. In some embodiments, the lanosteryl ester comprises a fatty acid having 22 carbon atoms. In some embodiments, the lanosteryl ester comprises a fatty acid having 23 carbon atoms. In some embodiments, the lanosteryl ester comprises a fatty acid having 24 carbon atoms. In some embodiments, the lanosteryl ester comprises a fatty acid having 25 carbon atoms. In some embodiments, the lanosteryl ester comprises a fatty acid having 26 carbon atoms. In some embodiments, the lanosteryl ester comprises a fatty acid having 27 carbon atoms. In some embodiments, the lanosteryl ester comprises a fatty acid having 28 carbon atoms. In some embodiments, the lanosteryl ester comprises a fatty acid having 29 carbon atoms. In some embodiments, the lanosteryl ester comprises a fatty acid having 30 carbon atoms.

Example lanosteryl esters include, but are not limited to, lanosteryl oleate, lanosteryl laurate, lanosteryl myristate, lanosteryl palmitate, lanosteryl stearate, lanosteryl isostearate, lanosteryl arachidate, lanosteryl behenate, lanosteryl lignocerate, lanosteryl cerotate, lanosteryl montanate, lanosteryl melissate, and lanosteryl nonanoate.

In some embodiments, the barrier repairing agent comprises a combination of cholesteryl esters and lanosteryl esters (e.g., as described herein). In some embodiments, the barrier repairing agent comprises a combination of C₁₀-C₃₀ cholesteryl esters and/or C₁₀-C₃₀ lanosteryl esters (e.g., as described herein), such combinations are also referred to as “c10-30 Cholesterol/lanosterol esters.”

In some embodiments, the barrier repairing agent comprises a polyglyceryl fatty acid ester. In some embodiments, the polyglyceryl fatty acid ester comprises a polyglyceryl moiety derived from 2 to 10 glycerin units, such as 3 to 10, 4 to 10, 5 to 10, 6 to 10, 7 to 10, 8 to 10 or 9 to 10 glycerin units. In some embodiments, the polyglyceryl fatty acid ester comprises a polyglyceryl moiety derived from 10 glycerin units. In some embodiments, the fatty acid ester is selected from sterate, oleate, ricinoleate, laurate, isostearate. In some embodiments, the polyglyceryl fatty acid is selected from polyglyceryl-2 stearate, polyglyceryl-2 oleate, polyglyceryl-2 isosterate, polyglyceryl-3 oleate, polyglyceryl-4 oleate, polyglyceryl-4 sterate, polyglyceryl-6 oleate, polyglyceryl-10 laurate, polyglyceryl-6 ricinoleate, polyglyceryl-10 linoleate, polyglyceryl-6 pentaoleate, polyglyceryl-3 dioleate, polyglyceryl-3 disterate, polyglyceryl-4 pentaoleate, polyglyceryl-6 dioleate, polyglyceryl-2 dioleate, polyglyceryl-10 trioleate, polyglyceryl-10 pentaoleate, polyglyceryl-10 septaoleate, polyglyceryl-10 tetraoleate, polyglyceryl-10 decaisostearate, polyglyceryl-10 decaoleate, polyglyceryl-10 mono, dioleate, polyglyceryl polyricinoleate. In some embodiments, the barrier repairing agent comprises polyglyceryl-10 decaisostearate.

In some embodiments, the barrier repairing agent is present in an amount ranging from 4% to 30% by weight of the composition. In some embodiments, the barrier repairing agent is present in an amount of 4% or more by weight, such as 5% or more, 6% or more, 7% or more, 8% or more, 9% or more, 10% or more, 15% or more, 20% or more, 25% or more, or 30% by weight of the composition.

In some embodiments, the barrier repairing agent comprises a mixture of C₁₀-C₃₀ cholesteryl esters and/or C₁₀-C₃₀ lanosteryl esters (e.g., as described herein). In some embodiments, the barrier repairing agent comprises a mixture of C₁₀-C₃₀ cholesteryl esters and/or C₁₀-C₃₀ lanosteryl esters and is present in an amount ranging from 4% to 30% by weight of the composition. In some embodiments, the mixture of C₁₀-C₃₀ cholesteryl esters and/or C₁₀-C₃₀ lanosteryl esters is present in an amount of 4% or more by weight, such as 5% or more, 6% or more, 7% or more, 8% or more, 9% or more, 10% or more, 15% or more, 20% or more, 25% or more, or 30% by weight of the composition.

In some embodiments, the barrier repairing agent is cholesteryl isostearate. In some embodiments, the barrier repairing agent is cholesteryl chloride. In some embodiments, the barrier repairing agent is cholesteryl nonanoate. In some embodiments, the barrier repairing agent is lanolin oil. In some embodiments, the barrier repairing agent is polyglyceryl-10 decaisostearate.

### 4.3. Hydrocarbon-Based Emollient

The anhydrous lipid composition of the present disclosure comprises one or more hydrocarbon-based emollients.

In one embodiment, the hydrocarbon-based emollient is selected from a linear higher alkane of animal, mineral or synthetic origin, a branched higher alkane of animal, mineral, or synthetic origin, and a combination thereof. The term “higher alkane” refers to an alkane have nine or more carbon atoms. In some embodiments, the hydrocarbon-based emollient comprises a linear or branched alkane having 9 carbon atoms. In some embodiments, the hydrocarbon-based emollient comprises a linear or branched alkane having 10 carbon atoms. In some embodiments, the hydrocarbon-based emollient comprises a linear or branched alkane having 11 carbon atoms. In some embodiments, the hydrocarbon-based emollient comprises a linear or branched alkane having 12 carbon atoms. In some embodiments, the hydrocarbon-based emollient comprises a linear or branched alkane having 13 carbon atoms. In some embodiments, the hydrocarbon-based emollient comprises a linear or branched alkane having 14 carbon atoms. In some embodiments, the hydrocarbon-based emollient comprises a linear or branched alkane having 15 carbon atoms. In some embodiments, the hydrocarbon-based emollient comprises a linear or branched alkane having 16 carbon atoms. In some embodiments, the hydrocarbon-based emollient comprises a linear or branched alkane having 17 carbon atoms. In some embodiments, the hydrocarbon-based emollient comprises a linear or branched alkane having 18 carbon atoms. In some embodiments, the hydrocarbon-based emollient comprises a linear or branched alkane having 19 carbon atoms. In some embodiments, the hydrocarbon-based emollient comprises a linear or branched alkane having 20 carbon atoms. In some embodiments, the hydrocarbon-based emollient comprises a linear or branched alkane having 21 carbon atoms. In some embodiments, the hydrocarbon-based emollient comprises a linear or branched alkane having 22 carbon atoms. In some embodiments, the hydrocarbon-based emollient comprises a linear or branched alkane having 23 carbon atoms. In some embodiments, the hydrocarbon-based emollient comprises a linear or branched alkane having 24 carbon atoms. In some embodiments, the hydrocarbon-based emollient comprises a linear or branched alkane having 25 carbon atoms. In some embodiments, the hydrocarbon-based emollient comprises a linear or branched alkane having 26 carbon atoms. In some embodiments, the hydrocarbon-based emollient comprises a linear or branched alkane having 27 carbon atoms. In some embodiments, the hydrocarbon-based emollient comprises a linear or branched alkane having 28 carbon atoms. In some embodiments, the hydrocarbon-based emollient comprises a linear or branched alkane having 29 carbon atoms. In some embodiments, the hydrocarbon-based emollient comprises a linear or branched alkane having 30 carbon atoms. In some embodiments, the hydrocarbon-based emollient comprises a range of linear and/or branched alkanes having 15 to 50 carbon atoms.

In some embodiments, the hydrocarbon-based emollient comprises a range of linear and/or branched alkanes having 15 to 30 carbon atoms. In some embodiments, the hydrocarbon-based emollient comprises a range of linear and/or branched alkanes having 15 to 20 carbon atoms. In some embodiments, the hydrocarbon-based emollient comprises a range of linear and/or branched alkanes having 13 to 20 carbon atoms. In some embodiments, the hydrocarbon-based emollient comprises a range of linear and/or branched alkanes having 13 to 15 carbon atoms.

In some embodiments, the hydrocarbon-based emollient is selected from mineral oil, a linear or branched C₁₀-C₃₀ alkane of animal origin, a linear or branched C₁₀-C₃₀ alkane of mineral, or synthetic origin, and any combination thereof.

In some embodiments, the hydrocarbon-based emollient comprises mineral oil. In some embodiments, the hydrocarbon-based emollient comprises a linear C₁₀-C₃₀ alkane of animal, mineral or synthetic origin, such as a linear C₁₀-C₁₅ alkane, or a linear C₁₃-C₁₅ alkane. In some embodiments, the hydrocarbon-based emollient comprises a branched C₁₀-C₃₀ alkane of animal, mineral or synthetic origin, such as a branched C₁₀-C₁₅ alkane, or a branched C₁₃-C₁₅ alkane.

In some embodiments, the hydrocarbon-based emollient comprises a linear or branched C₁₀-C₃₀ alkane of animal origin. In some embodiments, the linear or branched alkane of animal origin is a C₂₀-C₃₀ alkane, such as a C₂₅-C₃₀ alkane. Linear or branched C₁₀-C₃₀ alkanes of animal origin, include alkanes, such as squalane (hydrogenated squalene (sourced from shark liver oil)). Squalane has low acute toxicity and is not a significant human skin irritant or sensitizer.

In some embodiments, the hydrocarbon-based emollient comprises a linear or branched C₁₀-C₃₀ alkane of mineral or synthetic origin. In some embodiments, the linear or branched alkane of mineral or synthetic origin is a C₁₀-C₂₀ alkane, such as C₁₀-C₁₅ alkane, or C₁₃-C₁₅ alkane. Linear or branched C₁₀-C₃₀ alkane of mineral or synthetic origin, include alkanes such as hemisqualane, undecane, tridecane, isododecane, isohexadecane, hydrogenated polydecene, isoeicosane, hydrogenated didecene.

In some embodiments, the hydrocarbon-based emollient comprises isododecane. Due to its synthetic origin, isododecane is free of aromatics, and other undesirable impurities. It has low odor and very low toxicity. The high methyl-branching level of isododecane renders it particularly resistant to chemical attack or thermal degradation. Additionally, isododecane has a wide operating range both in terms of pressure and temperature.

In some embodiments, the hydrocarbon-based emollient is present in an amount ranging from 4% to 95% by weight of the composition, such as, e.g., 4% to 90%, 4% to 80%, 10% to 85%, 50% to 90%, 60% to 90%, 70% to 90%, or 80% to 95% by weight of the composition. In some embodiments, the one or more hydrocarbon-based emollients is present in an amount of 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% by weight of the composition.

In some embodiments, the one or more hydrocarbon-based emollients is selected from: mineral oil, squalane, c13-15 alkane, undecane, tridecane, isododecane, isohexadecane, hydrogenated polydecene, isoeicosane, hydrogenated didecene, and a combination thereof.

In some embodiments, the hydrocarbon-based emollient is mineral oil. In some embodiments, the hydrocarbon-based emollient is squalane. In some embodiments, the hydrocarbon-based emollient is a C₁₃-C₁₅ alkane, or any combination of C₁₃-C₁₅ alkanes. In some embodiments, the hydrocarbon-based emollient is undecane. In some embodiments, the hydrocarbon-based emollient is tridecane. In some embodiments, the hydrocarbon-based emollient is isododecane. In some embodiments, the hydrocarbon-based emollient is isohexadecane. In some embodiments, the hydrocarbon-based emollient is hydrogenated polydecene. In some embodiments, the hydrocarbon-based emollient is isoeicosane. In some embodiments, the hydrocarbon-based emollient is hydrogenated didecene.

In some embodiments, the hydrocarbon-based emollient is squalane, isododecane, or a combination thereof. In certain embodiments, the one or more hydrocarbon-based emollients is squalane. In certain embodiments, the one or more hydrocarbon-based emollients is isododecane. In certain embodiments, squalane is present in an amount of 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% by weight of the composition. In certain embodiments, isododecane is present in an amount of 4% or more, 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, or 95% or more by weight of the composition. In certain embodiments, the one or more hydrocarbon-based emollients includes 60% to 75% by weight of squalane and 1% to 15% by weight of isododecane.

In some embodiments, the hydrocarbon-based emollient has a boiling point range of from 150 to 380° C., such as e.g., 160 to 375° C., 165 to 330° C., 165 to 300° C., 165 to 280° C., 165 to 260° C., 165 to 240° C., 165 to 220° C., or 165 to 200° C. In some embodiments, the hydrocarbon-based emollient has a boiling point range of from 170 to 200° C., such as e.g., 170 to 195° C., 170 to 190° C., 170 to 185° C., or 170 to 180° C.

In some embodiments, the hydrocarbon-based emollient is not volatile at room temperature. In some embodiments, the hydrocarbon-based emollient is a mineral oil. In some embodiments, the hydrocarbon-based emollient is highly volatile and evaporate relatively quickly at room temperature. In certain embodiments, the hydrocarbon-based emollient is isododecane. In certain embodiments, the composition of the present disclosure includes a non-volatile hydrocarbon (e.g., squalane) along with a volatile one (e.g., isododecane) to get the right balance of emollience and lightness.

In some embodiments, the hydrocarbon-based emollient does not comprise a hydrocarbon-based oil of plant origin. In some embodiments, the hydrocarbon-based emollient does not comprise a triglyceride.

### 4.4. Additional Components

In some embodiments, the anhydrous lipid compositions of the present disclosure comprises one or more additional components.

In some embodiments, the additional component is water. In certain embodiments, the water is present in an amount of 2% or less, such as 1.5% or less, 1% or less, 0.5% or less, or 0.2% or less, or even less.

### 4.5. Containers

containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of the composition in a multi-use container which is pre-filled with a storage stable topical composition (e.g., as described herein).

Additional packaging for the container can be included. In some cases, the packaging provides a further barrier that prevents absorption of light and/or moisture from the atmosphere or surrounding environment.

### 4.6. Methods of Preparation

Also provided by this disclosure are processes for preparing the composition for storage that include preparation of any one of the subject formulations (e.g., as described herein), e.g., by dissolving niacinamide in a aqueous solvent with a primary oil-soluble antioxidant, a secondary oil-soluble antioxidant, a barrier repairing agent, and a hydrocarbon-based emollient, and one or more optionally additional components to provide a stable liquid composition capable of storage stability.

In some embodiments, the process includes combining:
a) 0.5% to 3% by weight of a primary oil-soluble antioxidant;
b) 0.5% to 5% by weight of a secondary oil-soluble antioxidant;
c) 4% to 30% by weight of a barrier repairing agent;
d) 4% to 95% by weight a hydrocarbon-based emollient; and
e) Less than 1% by weight water.

Also provided are product storage stable formulations produced by the process according to any one of the embodiments described herein.

### 5. EXAMPLES

### 5.1. Example 1: Exemplary Formulations

The exemplary formulations of Table 1 were prepared and assessed as having desirable properties.

TABLE 1
Components of Exemplary Compositions (% by weight)

| Formulation | 1 | 2 | 3 |
|---|---|---|---|
| Primary | 1% bakuchiol; | 1% bakuchiol; | 1% bakuchiol; |
| Antioxidant | 0.5% curcuma longa | 0.5% curcuma longa | 0.5% curcuma longa |
| | root extract | root extract | root extract |
| Secondary | 1% Tocopherol; | 1% Tocopherol; | 1% Tocopherol; |
| Antioxidant | 1% bis-ethylhexyl | 2% bis-ethylhexyl | 0.5% bis-ethylhexyl |
| | hydroxydimethoxy | hydroxydimethoxy | hydroxydimethoxy |
| | benzylmalonate | benzylmalonate | benzylmalonate |
| Barrier Repairing | 20% c10-30 | 20% c10-30 | 20% c10-30 |
| Agent | Cholesterol/lanosterol | Cholesterol/lanosterol | Cholesterol/lanosterol |
| | esters | esters | esters |
| Hydrocarbon-based | 71.5% Squalane | 65.5% Squalane | 67% Squalane |
| Emollient | 5% Isododecane | 10% Isododecane | 10% Isododecane |

### 5.2. Example 2: Storage Stability of Formulation 2

In order to test storage stability, testing was performed on formulation Formulation 2 in Table 1. 3 cycles of freeze/thaw testing, and 12 weeks of storage at 5° C., 25° C., 40° C., and 45° C. were performed while observing changes in color, odor, appearance and viscosity. No notable changes were observed, demonstrating storage stability, as shown in Table 2 below:

TABLE 2
Results of Storage Stability
Initial Stability Readings:

| Color | Odor | Appearance | pH | Viscosity | Sp. Gr. | % Solids |
|---|---|---|---|---|---|---|
| Straw | Characteristic | Clear, | N/A | N/A | 0.83 | N/A |
| | | non-viscous | | | | |
| | | liquid | | | | |

3 Cycles—Freeze/Thaw

| Cycle | Color | Odor | Appearance | pH | Viscosity |
|---|---|---|---|---|---|
| 1 | Conforms | Conforms | Conforms | N/A | N/A |
| 2 | Conforms | Conforms | Conforms | N/A | N/A |
| 3 | Conforms | Conforms | Conforms | N/A | N/A |
| | | 1 Week | 1 Month | 2 Months | 3 Months |
| Temp. | Date: | (Apr. 9, 2021) | (May 2, 2021) | (Jun. 2, 2021) | (Jul. 2, 2021) |
| 5° C. | Color | Conforms | Conforms | Conforms | Conforms |
| | Odor: | Conforms | Conforms | Conforms | Conforms |
| | Appearance | Conforms | Conforms | Conforms | Conforms |
| | pH: | N/A | N/A | N/A | N/A |
| | Viscosity: | N/A | N/A | N/A | N/A |
| 25° C. | Color: | Conforms | Conforms | Conforms | Conforms |
| (RT) | Odor: | Conforms | Conforms | Conforms | Conforms |
| | Appearance: | Conforms | Conforms | Conforms | Conforms |
| | pH: | N/A | N/A | N/A | N/A |
| | Viscosity: | N/A | N/A | N/A | N/A |
| 40° C. | Color: | Conforms | Conforms | Conforms | Conforms |
| w/ | Odor: | Conforms | Conforms | Conforms | Conforms |
| 75% | Appearance: | Conforms | Conforms | Conforms | Conforms |
| RH | PH: | N/A | N/A | N/A | N/A |
| | Viscosity: | N/A | N/A | N/A | N/A |
| 45° C. | Color: | Conforms | Conforms | Conforms | Conforms |
| | Odor: | Conforms | Conforms | Conforms | Conforms |
| | Appearance: | Conforms | Conforms | Conforms | Conforms |
| | pH: | N/A | N/A | N/A | N/A |
| | Viscosity: | N/A | N/A | N/A | N/A |

### 5.3. Example 3: Irritation and Sensitization Potential of Formulation 2

Human Repeat Insult Patch Testing (HRIPT) was conducted on formulation Formulation 2 in table Table 1 to demonstrate the absence of primary and cumulative irritation potential and skin sensitization potential under maximized conditions, controlling for product dosing and application site, and supervised by a dermatologist. The study duration was 6 weeks and 57 male and female subjects, ranging in age from 18 to 58.

Both investigational product and control were applied to patch test filter paper discs and then attached to the right or left back (scapular area) of the study subjects.

Induction Period: Applications were performed on Monday, Wednesday, and Friday, during 3 consecutive weeks. 48 hours, or 72 hours (on weekends), after product application, it was removed by trained technicians and the application site was assessed in order to check for the presence of clinical signs of irritation and sensitization. 9 applications were conducted during the induction period.

Rest Period: After the induction period, there was a minimum 10-day period when no product was applied.

Challenge test: Then, the challenge period started. One single application of the investigational product was carried out, followed by readings after 48 hours and 72 hours. Dermatological clinical assessments were made at the beginning and end of the study and subjects were supervised by a dermatologist throughout the study. 1 application was conducted during the challenge period.

Results: After undergoing dermatological testing, the product did not induce primary or cumulative irritation or skin sensitization in the study group, and was deemed safe under the study conditions. The results of the dermatological testing are shown in Table 3 below:

TABLE 3
Dermatological Testing of Formulation 2:

| | | Induction Period | | | | | | | | | | | | |
|---|---|---|---|---|---|---|---|---|---|---|---|---|---|---|
| | | Read- | Read- | Read- | Read- | Read- | Read- | Read- | Read- | | | | | |
| | | ing + | ing + | ing + | ing + | ing + | ing + | ing + | ing + | | | Challenge Period | | |
| | 1^{st} | 2^{nd} | 3^{rd} | 4^{th} | 5^{th} | 6^{th} | 7^{th} | 8^{th} | 9^{th} | | | 10^{th} | | |
| Subject | Appli- | appli- | appli- | appli- | appli- | appli- | appli- | Appli- | Appli- | Read- | Rest | Appli- | Read- | Read- |
| Number | cation | cation | cation | cation | cation | cation | cation | cation | cation | ing | Period | cation | ing | ing |
| 001 | 0 | 0 | 0 | 0 | 0 | 0 | 0 | 0 | 0 | 0 | No | 0 | 0 | 0 |
| 002 | 0 | 0 | 0 | 0 | 0 | 0 | 0 | 0 | 0 | 0 | Procedure | 0 | 0 | 0 |
| 003 | 0 | 0 | 0 | 0 | 0 | 0 | 0 | 0 | 0 | 0 | made | 0 | 0 | 0 |
| 004 | 0 | 0 | 0 | 0 | 0 | 0 | 0 | 0 | 0 | 0 | | 0 | 0 | 0 |
| 005 | 0 | 0 | 0 | 0 | 0 | F | 0 | 0 | 0 | 0 | | 0 | 0 | 0 |
| 006 | 0 | 0 | 0 | 0 | 0 | 0 | 0 | 0 | 0 | 0 | | 0 | 0 | 0 |
| 007 | 0 | 0 | 0 | 0 | 0 | 0 | 0 | 0 | 0 | 0 | | 0 | 0 | 0 |
| 008 | 0 | 0 | 0 | 0 | 0 | 0 | 0 | 0 | 0 | 0 | | 0 | 0 | 0 |
| 009 | 0 | 0 | 0 | 0 | 0 | 0 | 0 | 0 | 0 | 0 | | 0 | 0 | 0 |
| 011 | 0 | 0 | 0 | 0 | 0 | 0 | 0 | 0 | 0 | 0 | | 0 | 0 | 0 |
| 012 | 0 | 0 | 0 | 0 | 0 | 0 | 0 | 0 | 0 | 0 | | 0 | 0 | 0 |
| 013 | 0 | 0 | 0 | F | 0 | 0 | 0 | 0 | 0 | 0 | | 0 | 0 | 0 |
| 014 | 0 | 0 | 0 | 0 | 0 | 0 | 0 | 0 | 0 | 0 | | 0 | 0 | 0 |
| 015 | 0 | 0 | 0 | 0 | 0 | F | F/R | R | R | R | | R | R | R |
| 016 | 0 | F/R | R | R | R | R | R | R | R | R | | R | R | R |
| 017 | 0 | 0 | 0 | 0 | 0 | 0 | 0 | 0 | 0 | 0 | | 0 | 0 | 0 |
| 019 | 0 | 0 | 0 | 0 | 0 | 0 | F | F | 0 | 0 | | 0 | 0 | 0 |
| 020 | 0 | 0 | 0 | 0 | 0 | 0 | F | F | 0 | 0 | | 0 | 0 | 0 |
| 022 | 0 | 0 | 0 | 0 | 0 | 0 | F | F | 0 | 0 | | 0 | 0 | 0 |
| 023 | 0 | 0 | 0 | 0 | F | F/R | R | R | R | R | | R | R | R |
| 024 | 0 | 0 | 0 | 0 | 0 | 0 | 0 | 0 | 0 | 0 | | 0 | 0 | 0 |
| 025 | 0 | 0 | 0 | 0 | 0 | 0 | 0 | 0 | 0 | | | 0 | 0 | 0 |
| 028 | 0 | 0 | 0 | 0 | EA/R | R | R | R | R | R | | R | R | R |
| 029 | 0 | 0 | 0 | 0 | 0 | 0 | 0 | 0 | 0 | 0 | | 0 | 0 | 0 |
| 031 | 0 | 0 | 0 | 0 | 0 | 0 | F | F/R | R | R | | R | R | R |
| 032 | 0 | 0 | 0 | 0 | 0 | 0 | 0 | 0 | 0 | 0 | | 0 | 0 | 0 |
| 033 | 0 | 0 | 0 | 0 | 0 | 0 | 0 | 0 | 0 | 0 | | 0 | 0 | 0 |
| 034 | 0 | 0 | 0 | 0 | 0 | 0 | 0 | F | 0 | 0 | | 0 | 0 | 0 |
| 035 | 0 | 0 | 0 | 0 | 0 | 0 | 0 | 0 | 0 | 0 | | 0 | 0 | 0 |
| 036 | 0 | 0 | 0 | 0 | 0 | 0 | 0 | 0 | 0 | 0 | | 0 | 0 | 0 |

Caption:
X = Not Applied/Reading Not Performed
F = Absence
R = Removed from the Study
DK = darkening
DY = Dryness
F/R = Absence/Removed from the study
EA = Adverse Event
FS = Screening Failure checked after study
DP = Protocol Deviation
0 = No Reaction
1 = Mild Erythema
2 = Clear Erythema
3 = Erythema + Edema = Papules
4 = Erythema = Edem

### 5.4. Example 3: Antioxidant Defense of Formulation 2

As a measure of antioxidant activity, a 7-day minimal erythemal dose (MED) study was conducted on formulation 2 in table 1. Subjects had five test sites of 30 cm2 demarcated on the infrascapular region of the back. Test sites, untreated sites, and MED dose site were randomized per a computer-generated randomization scheme. ITA° value of each test site was determined by colorimetric method using a Chromameter. A total of 16 male and female subjects, 30-53 years of age, completed the clinical study.

Visit 1/Day 0: Photography was conducted of each test site and Chromameter measurements, with one replicate per subsite, taken to measure skin redness. Following evaluations and measurements, the test formula was applied to the designated treatment sites, while the untreated site remained without product application, and given a 5-minute dry time. Subjects were instructed to avoid UV exposure to the test area, apply other personal care products, and to avoid scratching or irritating the test sites. subjects were also instructed to avoid taking any medications or applying topical products that may mask or interfere with the study evaluations. Subjects were instructed to return the next day.

Visit 2-6/Day 1-5: After checking for compliance and adverse events, the test formula was again applied to the designated treatment sites, while the untreated site remained without product application and given a 5-minute dry time. Again, subjects were instructed to avoid UV exposure to the test area, apply other personal care products, and to avoid scratching or irritating the test sites. subjects were also instructed to avoid taking any medications or applying topical products that may mask or interfere with the study evaluations.

Visit 7/Day 6: After checking for compliance and adverse events, the test formula was again applied to the designated treatment sites, while the untreated site remained without product application and given a 5-minute dry time. During the dry time, a pre-MED was exposed on the previously demarcated test area. For the pre-MED, the range of UV doses were established using the estimated MEDu. Six (6) subsites centered by or close to the estimated MEDu were be exposed with incremental UV doses of 1.15×. MEDu was be estimated by ITA° range, and the associated dose used as a midpoint for the individual exposure sites. Following pre-MED exposure, the subjects were dismissed from the testing facility, and again instructed to avoid taking any medications or applying topical products that may mask or interfere with the study evaluations. Subjects were instructed to return the next day.

Visit 8/Day 7: After checking for compliance and adverse events, subjects were instructed to lie down for a minimum of 15 minutes to allow their skin to acclimate to the testing facility and to allow any demarcation left from clothing to subside. Following acclimatization, Chromameter measurements were conducted on UV-exposed sub-sites. Pre-MED sites were assessed for erythemal response based on the grading scale below. The first exposure-sub-site with a grade 1 score was used as the individual MEDu of the subject.

### Score Definitions:

0 No erythema present
0.5 Ambiguous erythema, and/or no clear border, and/or not filling more than 50% of exposure subsite
1 Perceptible unambiguous erythema with defined borders filling more than 50% of the exposure subsite
2 Moderate to intense erythema

Product-applied test sites were then irradiated using multiples of the previously determined MED. The sub-sites were exposed to 0.5×, 1.0×, 1.5×, 2.0×, 2.5× and 3.0× multiples of the previously determined MED. Following UV irradiation, the subjects were dismissed from the testing facility, and again instructed to avoid taking any medications or applying topical products that may mask or interfere with the study evaluations. Subjects were instructed to return the next day.

Visit 9/Day 8: After checking for compliance and adverse events, subjects were instructed to lie down for a minimum of 15 minutes to allow their skin to acclimate to the testing facility and to allow any demarcation left from clothing to subside. Following acclimatization, Chromameter measurements were conducted on all UV-exposed sub-sites. Irradiated sites were assessed for erythemal response based on the grading scale below, after which subjects were dismissed from the study.

### Score Definition

0 No erythema present
0.5 Ambiguous erythema, and/or no clear border, and/or not filling more than 50% of exposure subsite
1 Perceptible unambiguous erythema with defined borders filling more than 50% of the exposure subsite
2 Moderate to intense erythema

Results: The average MED on untreated sites was 255 joules per square meter. For sites treated with formulation 2 in table 1, 68.75% of subjects observed erythema in the 1.5×MED sub-site and 31.25% in the 1×MED sub-site, indicating significant increase in antioxidant defense for the treated sites. The average MED of sites treated with formula 2 in table 1 was 338 joules per square meter, with an average percentage change of 133% as compared to untreated sites

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A storage stable, topical, anhydrous, liquid composition comprising:
a. 0.5% to 3% by weight of a primary oil-soluble antioxidant comprising bakuchiol, Curcuma longa extract, or a combination thereof;
b. 0.5% to 5% by weight of a secondary oil-soluble antioxidant selected from: tocopherol, bis-ethylhexyl hydroxydimethoxy benzylmalonate, dimethylmethoxy chromanol, hexylresorcinol, and a combination thereof;
c. 4% to 30% by weight of a barrier repairing agent selected from: lanolin, cholesterol, a cholesterol derivative, lanosterol, a lanosterol derivative, a polyglyceryl fatty acid ester, and a combination thereof;
d. 4% to 95% by weight of a hydrocarbon-based emollient selected from: a linear higher alkane of animal, mineral or synthetic origin, a branched higher alkane of animal, mineral, or synthetic origin, and a combination thereof; and
e. Less than 1% by weight water.

2. The storage stable, topical, anhydrous, liquid composition of claim 1, wherein the primary oil-soluble antioxidant comprises about 1% by weight bakuchiol.

3. The storage stable, topical, anhydrous liquid composition of claim 1, wherein the primary oil-soluble antioxidant comprises about 0.5% by weight of Curcuma longa root extract.

4. The storage stable, topical anhydrous liquid composition of claim 1, wherein the secondary oil-soluble antioxidant comprises about 1% by weight tocopherol.

5. The storage stable, topical anhydrous liquid composition of claim 1, wherein the secondary oil-soluble antioxidant comprises about 0.5% to 2% by weight of bis-ethylhexyl hydroxydimethoxy benzylmalonate.

6. The storage stable, topical, composition of claim 1, wherein the barrier repairing agent comprises about 20% by weight of a mixture of C₁₀-C₃₀ Cholesteryl esters and C₁₀-C₃₀ lanosteryl esters.

7. The storage stable, topical, composition of claim 1, wherein the hydrocarbon-based emollient does not comprise a hydrocarbon-based oil of plant origin.

8. The storage stable, topical, composition of claim 7, wherein the hydrocarbon-based emollient does not comprise a triglyceride.

9. The storage stable, topical composition of claim 8, wherein the hydrocarbon-based emollient has a boiling point ranging from 150° C. to 380° C.

10. The storage stable, topical composition of claim 9, wherein the hydrocarbon-based emollient has a boiling point ranging from 150° C. to 300° C.

11. The storage stable, topical composition of claim 10, wherein the hydrocarbon-based emollient has a boiling point ranging from 165 to 200° C.
